(19) United States
(12) Patent Application Publication
(10) Pub. No.: US 2022/0296154 A1
(43) Pub. Date: Sep. 22, 2022

(54) METHOD FOR RAPIDLY DIAGNOSING A HEART CONDITION OF A PATIENT FROM ASSOCIATED ELECTROCARDIOGRAM ("ECG") DATA

(71) Applicant: Robert Dean MATOBA, Littleton, CO (US)

(72) Inventor: Robert Dean MATOBA, Littleton, CO (US)

(21) Appl. No.: 17/206,617

(22) Filed: Mar. 19, 2021

(51) Int. Cl. A61B 5/364; A61B 5/361; A61B 5/366; A61B 5/353; A61B 5/352; A61B 5/363

(52) U.S. Cl. CPC A61B 5/364; A61B 5/361; A61B 5/366; A61B 5/353; A61B 5/352; A61B 5/363

(57) ABSTRACT

A method for rapidly diagnosing a heart condition of a patient from associated ECG data presents a series of logic domains wherein corresponding logic tests solve in the alternative between successive logic domains to provide a mutually exclusive preliminary diagnosis. The present method enables even a novice emergency responder to be able to accurately present a preliminary diagnosis of a patient en route to urgent care.

### CROSS-REFERENCE TO RELATED APPLICATIONS

Not Applicable

### FEDERALLY SPONSORED RESEARCH OR DEVELOPMENT

Not Applicable

### INCORPORATION BY REFERENCE OF MATERIAL SUBMITTED ON A COMPACT DISK

Not Applicable

### COPYRIGHT NOTICE

Some portions of the disclosure of this patent document may contain material subject to copyright protection. The copyright owner has no objection to the facsimile reproduction by anyone of the patent document or ensuing disclosure as it appears on record at the Patent and Trademark Office, but otherwise reserves all copyright rights whatsoever.

### BACKGROUND OF THE INVENTION

According to the National Center for Chronic Disease Prevention and Health Promotion, Division for Heart Disease and Stroke Prevention, heart disease continues to be the leading cause of death for men, women, and people of most racial and ethnic groups in the United States. Heart disease presents multiple symptoms, heart failure may result from a number of different causes requiring different treatment options to prevent sudden death.

Monitoring a patient's cardiac rhythms not only provides insight into a patient's heart condition, but, as a vital sign used to monitor patient wellbeing in general, heart rate and rhythm provides useful insights for analysis considering patient health in general, including physical and emotion states. Heart vital signs are generated in multiple ways, but the most common, detailed view of a patient's cardiac cycles providing actionable information in real time is generated by measuring the changing voltage as the heart contracts, a process known as electrocardiography.

Electrocardiography is the process of producing an electrocardiogram (“ECG”), a graphical display of voltage versus time measured across the heart. The cardiac muscle produces measurable changes in voltage during each cardiac cycle (heartbeat), during depolarization and repolarization of the cardiac muscle each cardiac cycle. Depolarization starts in the sinoatrial node and spreads throughout the atrium, passes through the atrioventricular node, down into the bundle of His and into the Purkinje fibers, and then spreads into the ventricles. This regular pattern of depolarization produces a characteristic ECG tracing.

Each ECG, therefore, typically comprises three main components: the P-wave, the QRS complex, and the T wave. The P-wave represents an increasing voltage measurable as the atria depolarizes; the QRS complex represents changes in voltage occurrent as the ventricles depolarize; and the T wave represents the repolarization of the ventricles. Variances from this typical form evince potential cardiac abnormalities.

Cardiac irregularities often result in critical conditions wherein time is of the essence. There is little time to diagnose a patient undergoing cardiac arrest. What is needed is a method by which first responders can rapidly characterize cardiac irregularities to provide preliminary diagnoses en route to the hospital or emergency facility whereby medical staff may be expediently apprised of the potential diagnoses before taking charge of the patient.

### FIELD OF THE INVENTION

The present invention relates to a method for rapidly diagnosing a condition of a patient from associated ECG data. Indicator characteristics enable rapid determination of a particular heart problem from a single ECG readout when in transit.

### SUMMARY OF THE INVENTION

The present method for rapidly diagnosing a heart condition of a patient from associated electrocardiagram (“ECG”) data presents useful improvements in emergency management for patients suffering form heart trouble or from other illnesses for which heart rate signifies in disease presentation.

The present method enables emergency personnel to perform preliminary diagnoses during transportation of a patient en route to urgent care or otherwise ascertain in short order potential diseases and traumas associated with and signified by the heart. Where the steps of the method are followed, an accurate preliminary diagnosis is attained. Every minute counts during heart failure; a competent preliminary diagnosis before arriving at critical care may be the difference between life and death.

The present method for rapidly diagnosing a heart condition of a patient from associated ECG data provides a novel means of analyzing a cardiogram tracing of typically at least six seconds whereby each step in the method keys between logic domains in the alternative. Successive logic tests operative through a succession of associated logic domains key between alternative values. By ordering specific indicator characteristics into a mutually exclusive hierarchy, the present method enables emergency responders to determine reliable preliminary diagnoses through a series of discrete steps quickly and under pressure. Thus, in the midst of an emergency response, even a novice responder with little background in cardiography can, by application of the present method, ascertain a useful preliminary diagnosis that may mean the difference in saving a patient's life.

The present method for rapidly diagnosing a heart condition of a patient from associated ECG data therefore presents a discrete series of steps that leads to definite preliminary diagnoses actionable by medical staff upon arrival at urgent care. The present method views regularity and the rate of cardiac cycles (beats per minute, or “bpm”) and isolates key features of a cardiograph tracing of typically six seconds.

Heart rate is typically collected as a vital sign during transportation to care. Thus the first indication of vital signs yields the first step of the instant method—regularity of threshold heart rates that control a first order logic domain. If the heart rate is irregular, then a second order logic domain controls through successive tests to select between logic domains to a ninth order logic domain—to key out a single preliminary diagnosis. Logic domains under the domain of irregularity include the presence or absence of any of premature atrial contractions (“PACs”), premature junctional contractions (“PJCs”), or premature ventrical contractions (“PVCs”). Determination of additional elements illustrated in the cardiogram yields alternative logic tests used to key between mutually exclusive diagnoses. The order of steps executed when following the method has been devised to optimize accuracy of the preliminary diagnosis and represents a useful improvement in emergency care. By proceeding methodically by each step through the associated logic domains, an emergency responder is apt to determine a viable preliminary diagnosis.

If the heart rate is regular, then threshold rates (bpms) key between second order logic domains to control associated third to seventh order logic domains and ultimately yield a single preliminary diagnosis. Second order logic domains include threshold heart rates of ≤40 bpm, 41-59 bmp, 60-99 bpm, 100-149 bpm, and ≥150 bpm, from which separate logic domains depend to result in a final, mutually exclusive preliminary diagnosis.

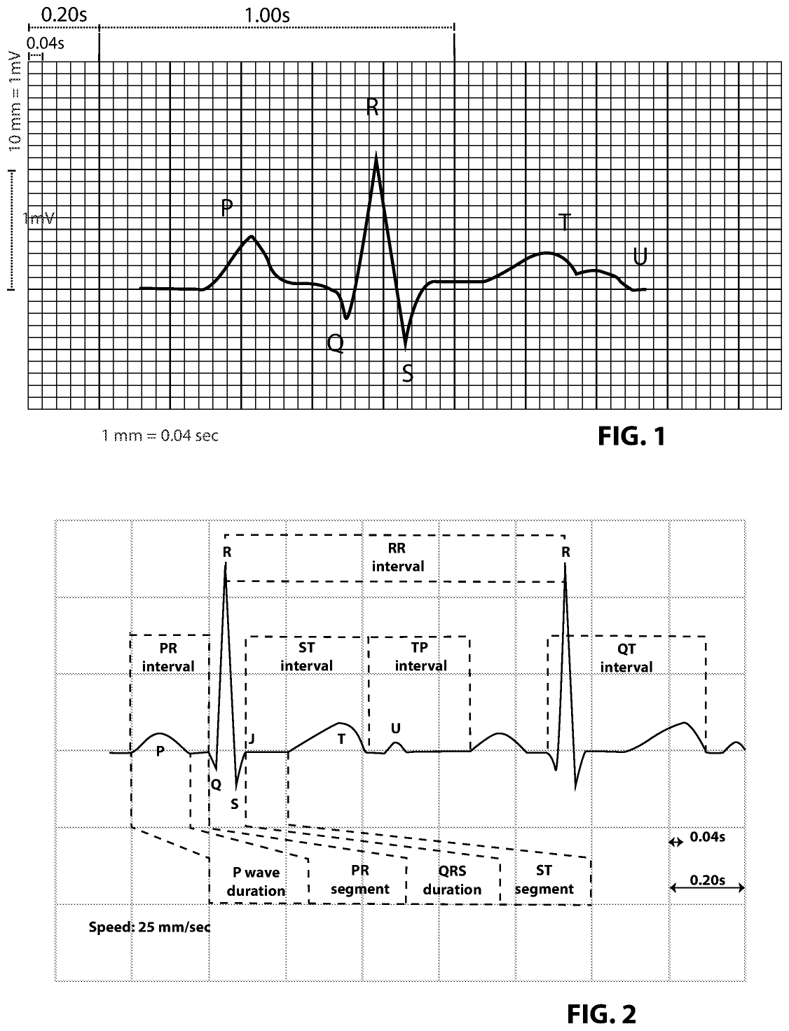

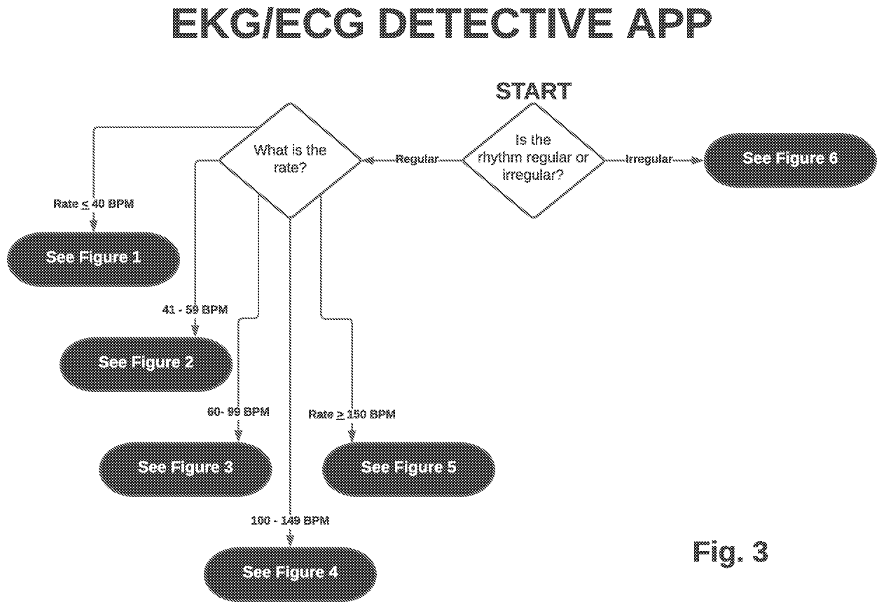

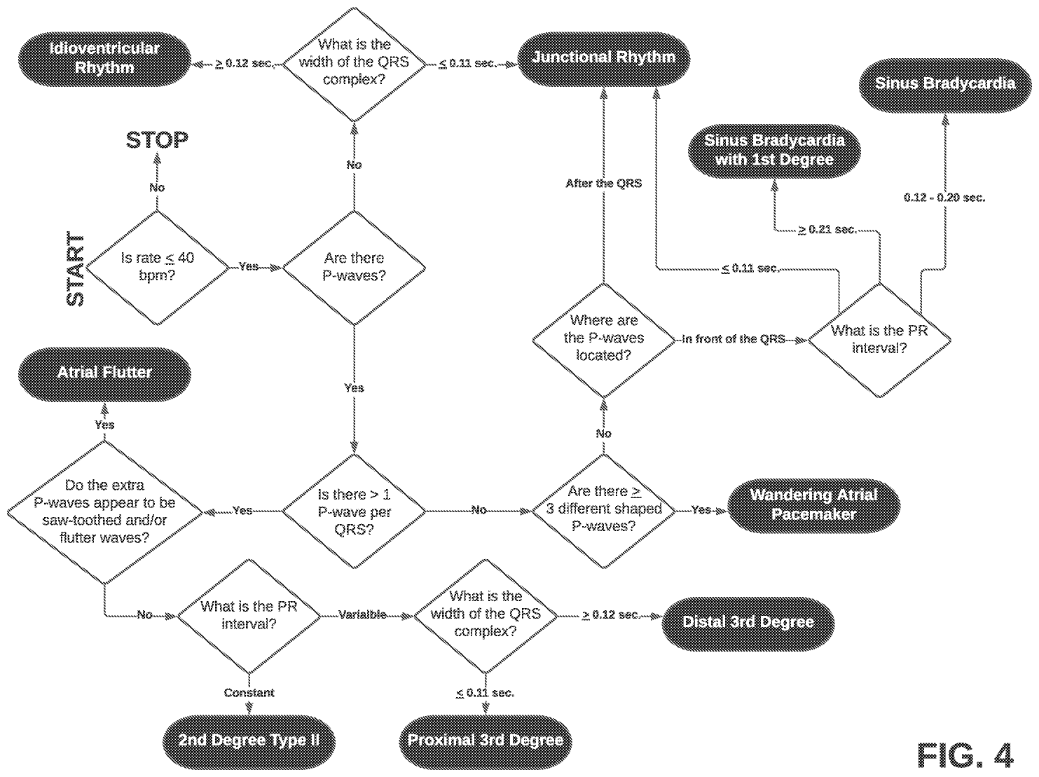

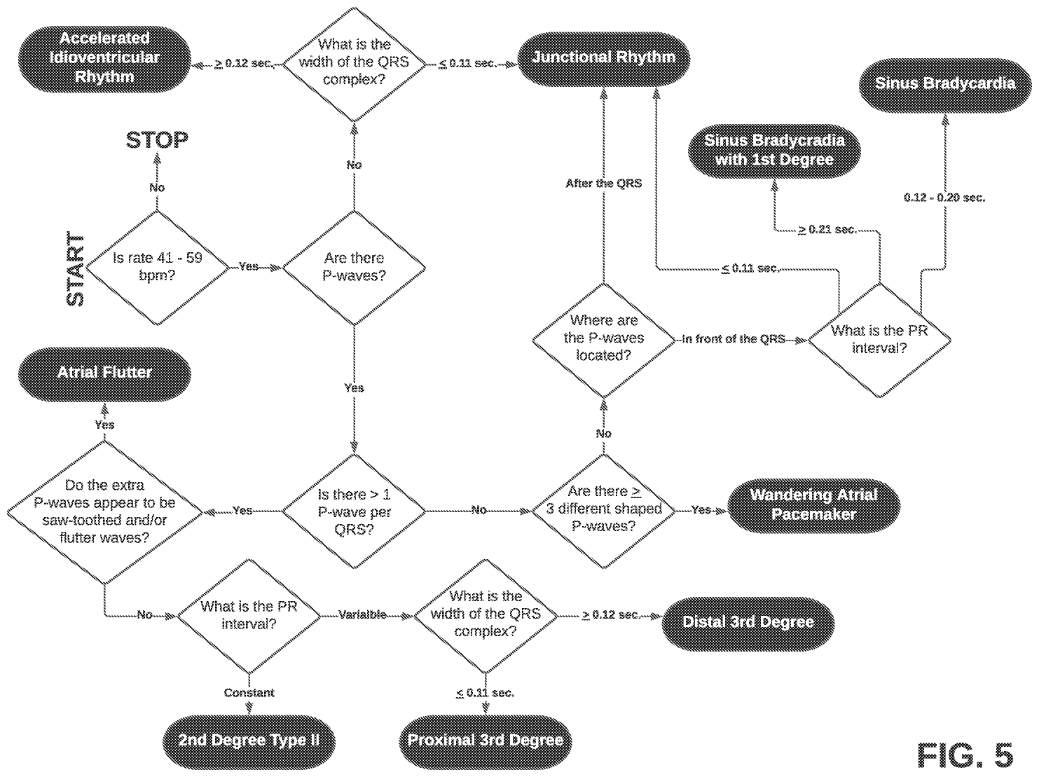

A cardiac cycle represents a typical tracing that is well recognized in the art and includes key features, designated alphabetically, with P, Q, R, S, T and U. See, e.g., FIG. 1. The interval between cardiac cycles corresponds to a heart rate (“bpm”) as represented on an electrocardiogram tracing where the horizontal axis represents intervals of time and the vertical axis represents voltage. Along the horizontal axis, the distance of 1 mm corresponds to 0.04 seconds (40 ms). Along the vertical axis, 10 mm equal 1 mV. Distances of 5 mm are indicated by bold lines, for ease of calculating heart rate, corresponding to 0.20 seconds (200 ms) along the horizontal axis and 0.5 mV along the vertical axis. Intervals corresponding to 1 second (25 mm) are indicated by lines extended to the edge of the tracing for ease of counting. See, e.g., FIGS. 1 and 2. Regularity or irregularity of a heartrate is therefore determinable in consideration of the intervals between P, Q, R, S, T, and U features displayed on the tracing (or represented in data captured from the fluctuating electromagnetic signal). Regularity corresponds to repeating cardiac cycles of similar frequency, within a range of relative norms. Irregularity corresponds to inconsistent intervals between cycles, evincing a chaotic and arrhythmic arrangement. As a first order logic domain, regularity or irregularity of the cardiac cycle keys to separate second order logic domains wherein a separate set of logic tests operate to key out a mutually exclusive preliminary diagnosis. Where a heart rate is regular—that is, evinces repeating periods of similar frequency within a range of relative norms—second order logic domains arise grouped by cycle frequency. Frequency ranges of 40 bpm, a slow heart rate, open subsequent logic tests between associated domains to key between idioventricular rhythm, junctional rhythm, atrial flutter, sinus brachycardia with 1^{st} degree, sinus brachycardia, wandering atrial pacemaker, distal 3^{rd} degree, proximal 3^{rd} degree, and 2^{nd} degree type II. Key features corresponding to abnormalities mutually exclude alternative diagnoses until a final preliminary diagnosis emerges as most probable. For example, absence of P-waves signals either idioventricular rhythm or junctional rhythm, depending on the width of the QRS complex. See, e.g., FIG. 3.

Where a regular heart rate second order logic domain consists of a rate between 41 and 59 bpm, subsequent logic domains arise selectable between logic tests to result in mutually exclusive diagnoses of accelerated idioventricular rhythm, junctional rhythm, sinus brachycardia, sinus brachycardia with 1^{st} degree, atrial flutter, wandering atrial pacemaker, distal 3^{rd} degree, proximal 3^{rd} degree, and 2^{nd} degree type II. See, e.g, FIG. 4. Similarly, a heart rate second order logic domain consisting of a rate between 60 and 99 bpm keys between five logic domains to result in mutually exclusive preliminary diagnoses of accelerated idioventricular rhythm, accelerated junctional rhythm, sinus rhythm, sinus rhythm with 1^{st} degree, wandering atrial pacemaker, atrial flutter, distal 3^{rd} degree, proximal 3^{rd} degree, and 2^{nd} degree type II. See, e.g., FIG. 5.

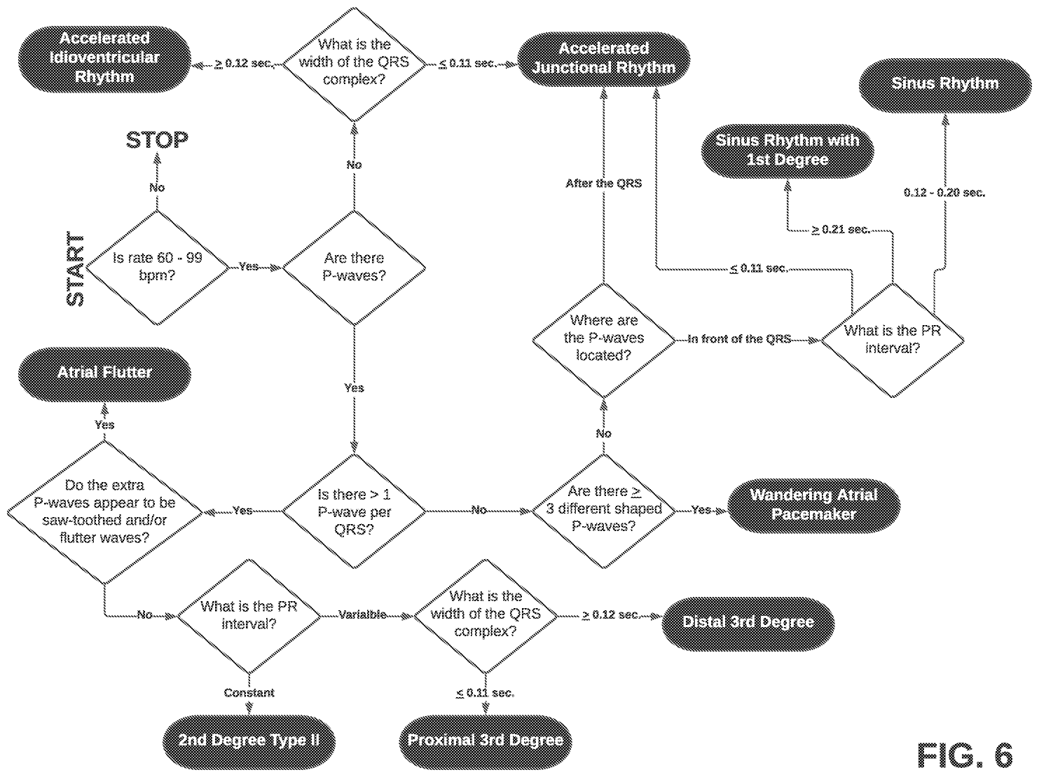

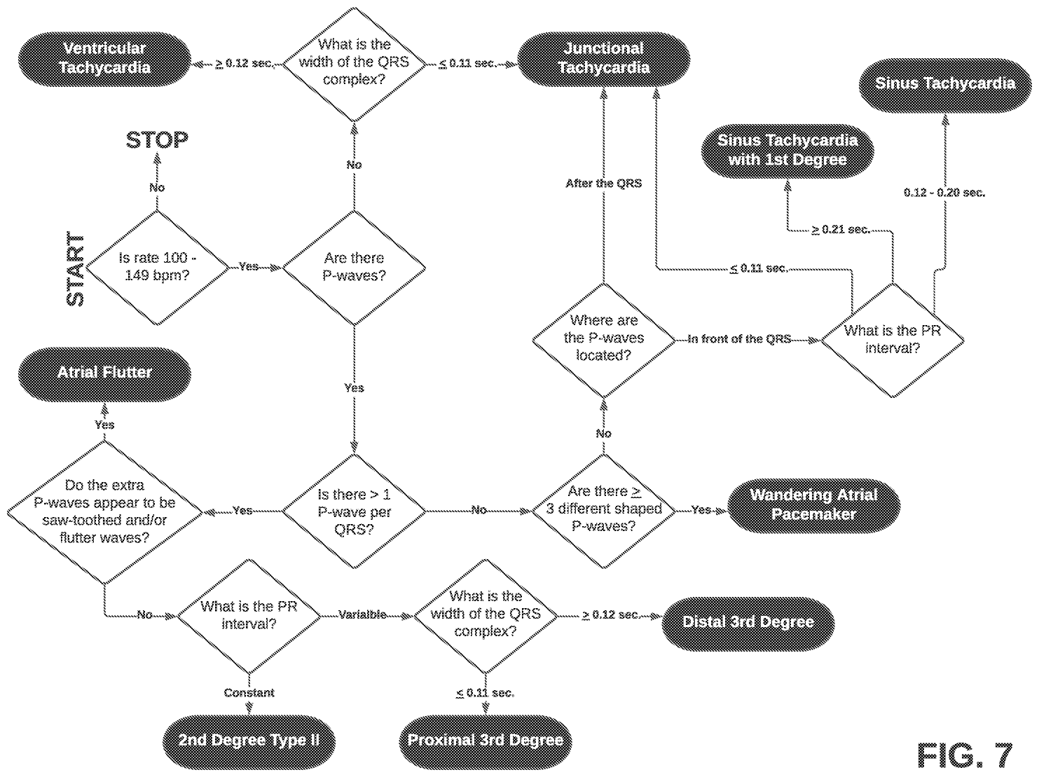

Heart rate second order logic domain consisting of a rate between 100 and 149 bpm keys to ventricular tachycardia, junctional tachycardia, sinus tachycardia, sinus tachycardia with 1^{st} degree, wandering atrial pacemaker, atrial flutter, distal 3^{rd} degree, proximal 3^{rd} degree, and 2^{nd} degree type II. See, e.g., FIG. 6. Heart rates above 150 bpm operate in a third order logic domain only, wherein the width of the QRS complex is used as a test indicator to select between ventricular tachycardia and supraventricular tachycardia. See, e.g., FIG. 7.

It should be noted that some indicator characteristics are universal where the second order logic domain consists of regular heart rate. Where there is more than one P-wave per QRS complex, for example, and the P-waves are saw-toothed and/or represent flutter waves, atrial flutter is signified irrespective of the heart rate. Where the P-wave per cycle comprises at least three different shapes, then a wandering atrial pacemaker is signified. However, these diagnoses key from fourth and fifth order logic domains respectively in order to mutually exclude previous potential diagnoses first. Therefore, the ordering of the steps comprising the method is important; in order to efficiently dispose of diagnoses, the steps proceed from common logic domains through a series of logic tests to exclude alternatives and arrive at an accurate preliminary diagnosis in optimized order. Where a patient is suffering from heart problems, a process created to save time and optimize diagnoses to focus treatment options represents a useful improvement in the art. It saves lives.

Irregular heart rates key through eight successive logic domains to mutually exclude preliminary diagnoses of premature junctional contractions (“PJCs”), premature ventricular contractions (“PVCs”), premature atrial contractions (“PACs”), sinus arrest, sinus exit block, sinus arrest with 1^{st} degree, sinus exit block with 1^{st} degree, atrial flutter, 2^{nd} degree type I, 2nd degree type II, sinus arrhythmia, sinus arrythmias with 1^{st} degree, wandering atrial pacemaker, atrial fibrillation, and ventricular fibrillation. Thus has been broadly outlined the more important features of the present method for rapidly diagnosing a heart condition of a patient from associated ECG data so that the detailed description thereof that follows may be better understood and in order that the present contribution to the art may be better appreciated.

Objects of the present method for rapidly diagnosing a heart condition of a patient from associated ECG data, along with various novel features that characterize the invention are particularly pointed out in the claims forming a part of this disclosure. For better understanding of the method for rapidly diagnosing a heart condition of a patient from associated ECG data, its operating advantages and specific objects attained by its uses, please refer to the accompanying drawings and description.

### BRIEF DESCRIPTION OF THE DRAWINGS

### Figures

FIG. 1 is a diagrammatic view of an electrocardiograph tracing of voltage over time identifying the characteristic waveforms designated in the art by the letters P, Q, R, S, T, and U.

FIG. 2 is a diagrammatic view of an electrocardiograph tracing illustrating the various sections of the waveform applicable to logic tests set forth in the present method.

FIG. 3 is a flow diagram illustrating the first order logic domain to which the present method applies to key between FIGS. 4, 5, 6, 7, 8, and 9.

FIG. 4 is a flow diagram illustrating the second order logic domain consisting of rate 40 bpm initiating a series of logic tests to key through successive logic domains to present mutually exclusive preliminary diagnoses between a series of logic pathways.

FIG. 5 is a flow diagram illustrating the second order logic domain consisting of rate between 41 and 59 bpm initiating a series of logic tests to key through successive logic domains to present mutually exclusive preliminary diagnoses between a series of logic pathways.

FIG. 6 is a flow diagram illustrating the second order logic domain consisting of rate between 60 and 99 bpm initiating a series of logic tests to key through successive logic domains to present mutually exclusive preliminary diagnoses between a series of logic pathways.

FIG. 7 is a flow diagram illustrating the second order logic domain consisting of rate between 100 and 149 bpm initiating a series of logic tests to key through successive logic domains to present mutually exclusive preliminary diagnoses between a series of logic pathways.

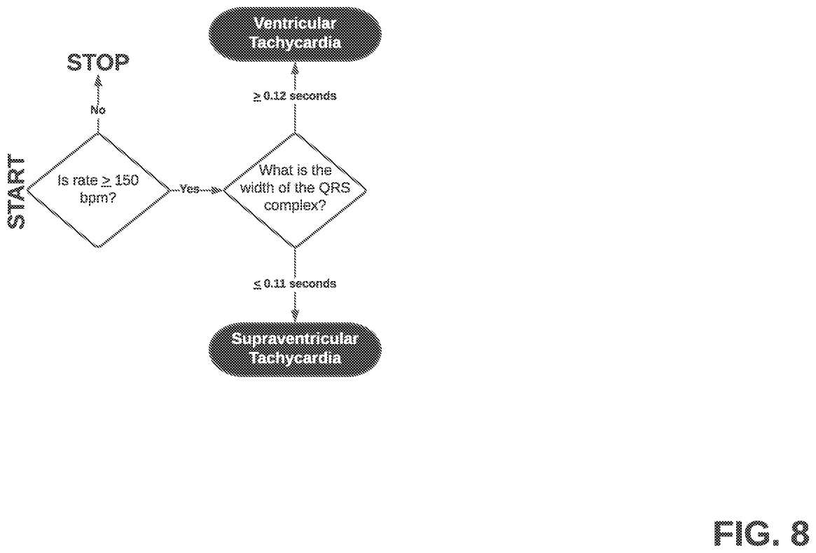

FIG. 8 is a flow diagram illustrating the second order logic domain consisting of rate 150 bpm initiating a third order logic domain to tests to key between mutually exclusive preliminary diagnoses.

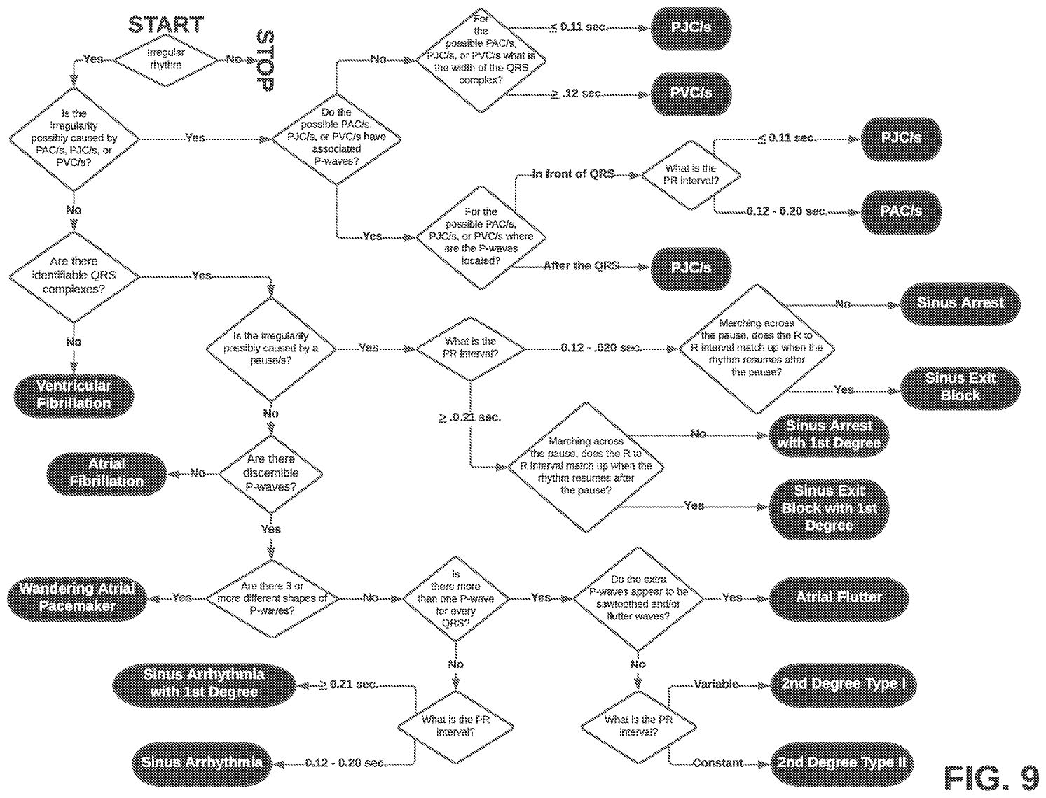

FIG. 9 is a flow diagram illustrating the second order logic domain consisting of rate irregular rhythm initiating a series of logic tests to key through successive logic domains to present mutually exclusive preliminary diagnoses between a series of logic pathways.

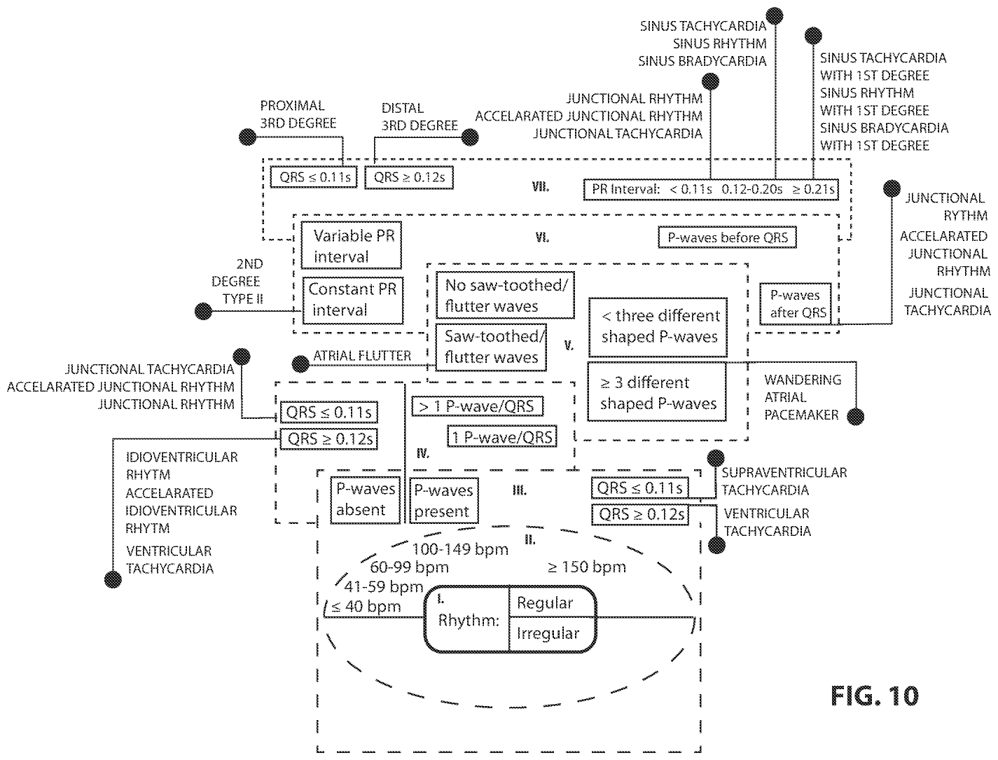

FIG. 10 is a diagrammatic view of the orders of logic domains operative from a first order logic domain selecting for regular rhythm inclusive of seven orders. Selecting in the alternative through each domain yields mutually exclusive diagnoses.

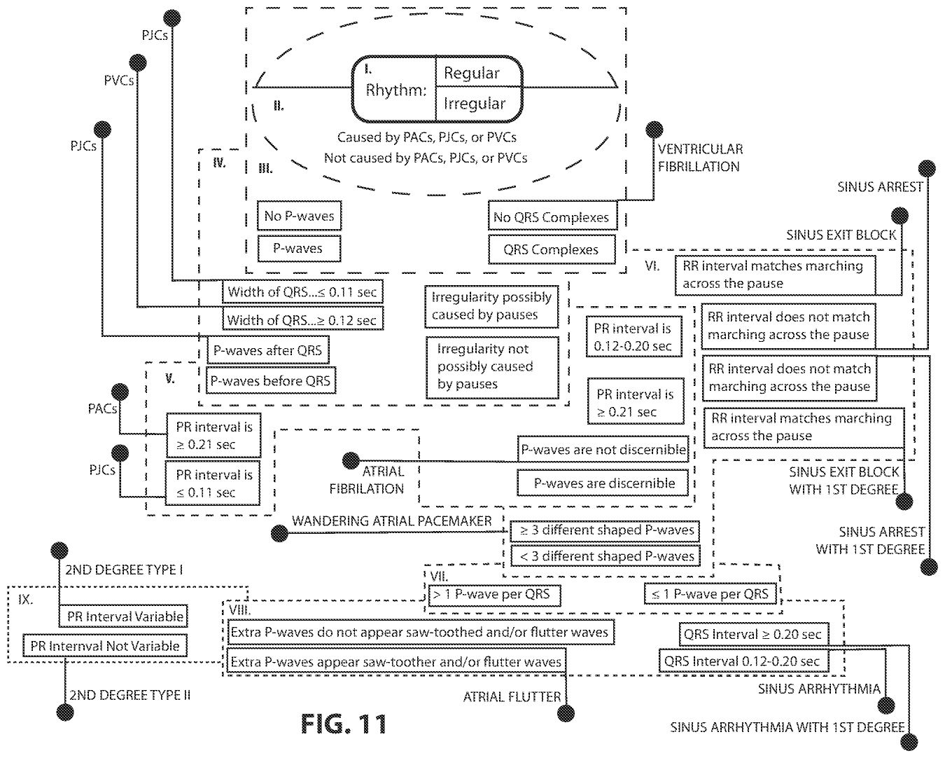

FIG. 11 is a diagrammatic view of the orders of logic domains operative from a first order logic domain selecting for irregular rhythm inclusive of XX orders. Selecting in the alternative through each domain yields mutually exclusive diagnoses.

### DETAILED DESCRIPTION OF THE DRAWINGS

With reference now to the drawings, and in particular FIGS. 1 through 11 thereof, example of the instant method for rapidly diagnosing a heart condition of a patient from associated ECG data employing the principles and concepts of the present method for rapidly diagnosing a heart condition of a patient from associated ECG data and generally designated by the reference number 10 will be described.

FIG. 1 illustrates a standard cardiac cycle as represented by an ECG tracing. The tracing shown illustrates 2.12 seconds and portrays the distinctive waveform common to cardiac cycles, comprising a P-wave (atrial depolarization), a QRS complex (ventricular depolarization), a T wave (ventricular repolarization). The U wave (Purkinje repolarization) is often visible, but not always. The relationships and form of these cardiac processes across time are usable to signify particular diagnoses associated with various conditions and as vital signs. As shown in FIG. 1, the wave form is traced upon a background grid where each line represents 0.04 seconds (1 mm in horizontal distance) and each bold line represents 0.20 seconds (5 mm in horizontal distance). Amplitude of the waveform is measured along the vertical axis wherein 10 mm in vertical distance corresponds to 1 mV.

FIG. 2 illustrates the various sections of the overall waveform contemplated in the present method, variations in which sections enable appropriate selection between logic domains to mutually exclude diagnoses and ultimately arrive at a preliminary diagnosis.

The duration of the P-wave (atrial depolarization) is indicated by the change of slope of the waveform off of the baseline and its subsequent return to the baseline. The PR segment (time between atrial depolarization and ventricular depolarization) is measurable between the end of the P-wave and the onset of the QRS complex. QRS duration (ventricular depolarization) ends with the return to baseline at J, and the ST segment (time taken for ventricles to begin to repolarize) continues until the change in slope indicating initiation of the T wave (ventricular repolarization). Various intervals are shown that correspond to the rate and regularity of heartbeats, including the PR interval, the RR interval (between successive beats), the ST interval and the TP interval. QT interval is also shown.

FIG. 3 is a flow diagram showing the first order logic domain in which the present method operates. The ECG tracing is considered or captured and the first logic test is applied, to solve alternatively between regularity or irregularity of the rhythm displayed. Irregularity in this sense is unrepeating patterns and/or irregular changes in rate between identifiable portions of the waveform. If irregularity is apparent, the second order logic domain represented in initiating the steps shown in FIG. 9 is undertook. If the rhythm is regular, a second order logic test selects between rates ≤40 bpm (see FIG. 4), 41-49 bpm (see FIG. 5), 60-99 bpm (see FIG. 6), 100-149 bpm (see FIG. 7) and ≥150 bpm (see FIG. 8).

Thus the initial step in the present method it to determine regularity or irregularity of the patient's cardiac cycles.

TABLE 1
First Order and Second Order Logic Domains

| ORDER | TEST | |
|---|---|---|
| I | a. | Regular rhythm: II(a) |
| | b. | Irregular rhythm: II(b) |
| II(a) | a. | Rate ≤40 bpm: TABLE 2 (FIG. 4) |
| | b. | Rate 41-59 bpm: TABLE 3 (FIG. 5) |
| | c. | Rate 60-99 bpm: TABLE 4 (FIG. 6) |
| | d. | Rate 100-149 bpm: TABLE 5 (FIG. 7) |
| | e. | Rate ≥150 bpm: TABLE 6 (FIG. 8) |
| II(b) | a. | Irregularity possibly caused by PACs, PJCs, |
| | | or PVCs: TABLE 7 (FIG. 9) |
| | b. | Irregularity not possibly caused by PACs, PJCs, |
| | | or PVCs: TABLE 7 (FIG. 9) |

We will now treat of each associated logic test operative in successive logic domains. Each test solves in the alternative between succedent domains to yield a mutually exclusive diagnosis via a series of associated steps comprising the method.

FIG. 4: III Order Logic Domain: Rate≤40 bpm

Where the cardiac cycle is regular and rate is less that ≤40 bpm, a third order logic domain consists of presence or absence of P-waves. If the third order logic test solves to no P-waves apparent, then the width of the QRS complex presents a fourth order logic domain to key between the alternative preliminary diagnoses, idioventricular rhythm (QRS complex width is ≥0.12 seconds, that is shown over at least 3 mm of the tracing), or junctional rhythm (QRS complex ≤0.11 seconds, that is shown over less than 3 mm of tracing). No further test is necessary.

Where the third order logic test solves that there are P-waves operative within the third order logic domain, then a fourth order logic domain opens to test in the alternative whether or not there are more than one P-wave per QRS complex. Where the fourth order logic test solves that there are more than one P-wave per QRS complex, a fifth order logic domain opens to test in the alternative whether or not the extra P-waves are saw-toothed and/or flutter waves (that is, evidence of looped electrical activity to depolarize the atrium). If the fifth order logic tests solves that the extra P-waves are saw-toothed and/or flutter waves, then atrial flutter is the preliminary diagnosis keyed out at the fifth order logic domain. No further test is necessary.

Continuing along this logic chain in the associated fifth order logic domain, where the fifth order logic test solves that the extra P-waves do not appear to be saw-toothed and/or flutter waves, then a sixth order logic domain opens for a sixth order logic test in the alternative to determine whether or not the PR interval is constant or variable. Where the sixth order logic test solves that the PR interval is determined to be constant, then 2^{nd} degree type II is the preliminary diagnosis keyed out at the sixth order logic domain. No further test is necessary.

Alternatively, where the sixth order logic test solves that the PR interval is variable, a seventh order logic domain opens to test whether the QRS complex has a width of 0.11 seconds (<3 mm) or, alternatively, a width of ≥0.12 seconds (≥3 mm). If the seventh order test solves that the QRS complex has a width of ≤0.11 seconds, then proximal 3^{rd} degree is the preliminary diagnosis keyed out at the seventh order logic domain and no further test is necessary. Alternatively, if the seventh order logic test solves that the QRS complex has a width of ≥0.12 seconds, then distal 3^{rd} degree is the preliminary diagnosis keyed out at the seventh order logic domain. No further test is necessary.

Returning now to the fourth order logic test of whether or not there is >1 P-wave per QRS, solving in the alternative (at most 1 P-wave per QRS) opens a fifth order logic domain to test whether or not there are three or more different shaped P-waves on the tracing. If the fifth order logic test solves that there are at least 3 different shaped P-waves on the tracing, then the preliminary diagnosis is wandering atrial pacemaker keyed out at the fifth order logic domain. No further test is necessary.

Alternatively, if the fifth order logic test solves that there are not three or more different shaped P-waves evident on the tracing, a sixth order logic domain opens to test between locations of the P-waves in the alternative. Where the sixth order logic test solves that the P-waves are located after the QRS complex, junctional rhythm is the preliminary diagnosis keyed out at the sixth order logic domain and no further test is necessary. Alternatively, where the sixth order logic test solves that P-waves are located in front of the QRS complex, then a seventh order logic domain opens wherein the PR interval controls between three possible mutually exclusive outcomes: viz., a PR interval of ≤0.11 seconds results in a preliminary diagnosis of junctional rhythm keyed out at the seventh order logic domain. No further test is necessary. A PR interval of 0.12-020 seconds results in a preliminary diagnosis of sinus bradycardia. No further test is necessary. And a PR interval of ≥0.21 seconds results in a preliminary diagnosis of sinus bradycardia with 1^{st} degree.

Thus, all potential diagnoses for regular heart rates of ≤40 bpm are mutually excluded by following the steps of the present method to present a single preliminary diagnosis by logic tests selecting in the alternative.

The orders of logic domains and associated logic tests under the regular rhythm domain solving to ≤40 bpm are summarized below in Table 2.

TABLE 2
Orders of Logic Domains Under Rate ≤40 bpm

| ORDER | TEST | |
|---|---|---|
| III | a. | Presence of P-waves: IV(a) |
| | b. | Absence of P-waves: IV (b) |
| IV(a) | a. | Width of QRS complex ≥012 sec: IDIOVENTRICULAR |
| | | RHYTHM |
| | b. | Width of QRS complex ≤0.11 sec: JUNCTIONAL |
| | | RHYTHM |
| IV(b) | a. | >1 P-wave/QRS: V(a) |
| | b. | 1 P-wave/QRS: V(b) |
| V(a) | a. | Extra P-waves are saw-toothed and/or |
| | | flutter waves: ATRIAL FLUTTER |
| | b. | Extra P-waves are not saw-toothed and/ |
| | | or flutter: VI(b)(i) |
| V(b) | a. | There are at least 3 different shaped |
| | | P-waves: WANDERING ATRIAL PACEMAKER |
| | b. | There are less than 3 different shaped |
| | | P-waves: VI(b)(ii) |
| VI(b)(i) | a. | PR interval is constant: 2^{ND} DEGREE TYPE II |
| | b. | PR interval is variable: VII(b)(i) |
| VI(b)(ii) | a. | P-waves located after QRS: JUNCTIONAL |
| | | RHYTHM |
| | b. | P-waves located before QRS: VII(b)(ii) |
| VII(b)(i) | a. | Width of QRS complex ≥012 sec: DISTAL |
| | | 3^{RD} DEGREE |
| | b. | Width of QRS complex ≤0.11 sec: PROXIMAL |
| | | 3^{RD} DEGREE |
| VII(b)(ii) | a. | PR interval is ≤0.11 sec: JUNCTIONAL RHYTHM |
| | b. | PR interval is 0.12-0.20 sec: SINUS BRADYCARDIA |
| | c. | PR interval is ≥0.21 sec: SINUS BRADYCARDIA WITH |
| | | 1^{ST} DEGREE |

FIG. 5: III Order Logic Domain: Rate 41-59 bpm

Where the cardiac cycle is regular and rate is between 41 and 59 bpm, a third order logic domain consists of presence or absence of P-waves. If the third order logic test solves to no P-waves apparent, then the width of the QRS complex presents a fourth order logic domain to key between the alternative preliminary diagnoses, idioventricular rhythm (QRS complex width is ≥0.12 seconds, that is shown over at least 3 mm of the tracing), or junctional rhythm (QRS complex ≤0.11 seconds, that is shown over less than 3 mm of tracing). No further test is necessary.

Where the third order logic test solves that there are P-waves operative within the third order logic domain, then a fourth order logic domain opens to test whether or not there are more than one P-wave per QRS complex. Where the fourth order logic test solves that there are more than one P-wave per QRS complex, a fifth order logic domain opens to test in the alternative whether the extra P-waves are saw-toothed and/or flutter waves (that is, evidence of looped electrical activity to depolarize the atrium). If the extra P-waves are saw-toothed and/or flutter waves, then atrial flutter is the preliminary diagnosis, keyed out at the fifth order logic domain. No further test is necessary.

Continuing along this logic chain in the associated fifth order logic domain, where the fifth order logic test solves that the extra P-waves do not appear to be saw-toothed and/or flutter waves then a sixth order logic domain opens for a sixth order logic test in the alternative to determine whether the PR interval is constant or variable. Where the PR interval is constant, then 2^{nd} degree type II is the preliminary diagnosis, keyed out at the sixth order logic domain. No further test is necessary.

Alternatively, where the PR interval is variable, a seventh order logic domain opens to test whether the QRS complex has a width of ≤0.11 seconds (<3 mm) or ≥0.12 seconds (≥3 mm). If the QRS complex has a width of 0.11 seconds, then proximal 3^{rd} degree is the preliminary diagnosis keyed out at the seventh order logic domain and no further test is necessary. Alternatively, if the QRS complex has a width of ≥0.12 seconds, then distal 3^{rd} degree is the preliminary diagnosis keyed out at the seventh order logic domain and no further test is necessary.

Returning now to the fourth order logic test of whether or not there is >1 P-wave per QRS, solving in the alternative (at most 1 P-wave per QRS) opens a fifth order logic domain to test whether or not there are three or more different shaped P-waves on the tracing. If the fifth order logic test solves that there are at least 3 different shaped P-waves on the tracing, then the preliminary diagnosis is wandering atrial pacemaker, keyed out at the fifth order logic domain. No further test is necessary.

Alternatively, if the fifth order logic test solves that there are not three or more different shaped P-waves evident on the tracing, a sixth order logic domain opens to test between location of the P-waves in the alternative. Where the sixth order logic test solves that the P-waves are located after the QRS complex, junctional rhythm is the preliminary diagnosis, keyed out at the sixth order logic domain and no further test is necessary. Alternatively, where the sixth order logic test solves that the P-waves are determined to be located in front of the QRS complex, then a seventh order logic domain opens wherein the PR interval controls between three possible mutually exclusive outcomes: viz., a PR interval of ≤0.11 seconds results in a preliminary diagnosis of junctional rhythm keyed out at the seventh order logic domain. No further test is necessary. A PR interval of 0.12-020 seconds results in a preliminary diagnosis of sinus bradycardia. No further test is necessary. And a PR interval of ≥0.21 seconds results in a preliminary diagnosis of sinus bradycardia with 1^{st} degree.

Thus, all potential diagnoses for regular heart rates of 41-59 bpm are mutually excluded by following the steps of the present method to present a single preliminary diagnosis by test selecting in the alternative.

The orders of logic domains and associated logic tests under the regular rhythm logic domain solving to 41-59 bpm are summarized below in Table 3.

TABLE 3
Orders of Logic Domains Under Rate 41-59 bpm

| ORDER | TEST | |
|---|---|---|
| III | a. | Presence of P-waves: IV(a) |
| | b. | Absence of P-waves: IV (b) |
| IV(a) | a. | Width of QRS complex ≥012 sec: ACCELARATED |
| | | IDIOVENTRICULAR RHYTHM |
| | b. | Width of QRS complex ≤0.11 sec: JUNCTIONAL |
| | | RHYTHM |
| IV(b) | a. | >1 P-wave/QRS: V(a) |
| | b. | 1 P-wave/QRS: V(b) |
| V(a) | a. | Extra P-waves are saw-toothed and/or flutter |
| | | waves: ATRIAL FLUTTER |
| | b. | Extra P-waves are not saw-toothed and/or flutter: VI(b)(i) |
| V(b) | a. | There are at least 3 different shaped P-waves: |
| | | WANDERING ATRIAL PACEMAKER |
| | b. | There are less than 3 different shaped P-waves: VI(b)(ii) |
| VI(b)(i) | a. | PR interval is constant: 2^{ND} DEGREE TYPE II |
| | b. | PR interval is variable: VII(b)(i) |
| VI(b)(ii) | a. | P-waves located after QRS: JUNCTIONAL RHYTHM |
| | b. | P-waves located before QRS: VII(b)(ii) |
| VII(b)(i) | a. | Width of QRS complex ≥012 sec: DISTAL 3^{RD} DEGREE |
| | b. | Width of QRS complex ≤0.11 sec: PROXIMAL |
| | | 3^{RD} DEGREE |
| VII(b)(ii) | a. | PR interval is ≤0.11 sec: JUNCTIONAL RHYTHM |
| | b. | PR interval is 0.12-0.20 sec: SINUS BRADYCARDIA |
| | c. | PR interval is ≥0.21 sec: SINUS BRADYCARDIA WITH |
| | | 1^{ST} DEGREE |

FIG. 6: III Order Logic Domain: Rate 60-99 bpm

Logic domains opening for rate between 60 and 99 seconds proceed in like manner as above, however different mutually exclusive diagnoses are presented accordant with the higher heart rate.

Thus, where the cardiac cycle is regular and rate is between 60 and 99 bpm, a third order logic domain consists of presence or absence of P-waves. Where the third order logic test solves to no P-waves apparent, then the width of the QRS complex presents a fourth order logic domain to key between alternative preliminary diagnoses: accelerated idioventricular rhythm (QRS complex width is ≥0.12 seconds) or accelerated junctional rhythm (QRS complex ≤0.11 seconds). No further test is necessary.

Where the third order logic test solves that there are P-waves operative within the third order logic domain, then a fourth order logic domain opens to test in the alternative whether or not there are more than one P-wave per QRS complex. Where the fourth order logic test solves that there are more than one P-wave per QRS complex, a fifth order logic domain opens to test in the alternative whether or not the extra P-waves are saw-toothed and/or flutter waves (that is, evidence of looped electrical activity to depolarize the atrium). If the fifth order logic test solves that the extra P-waves are saw-toothed and/or flutter waves, then atrial flutter is the preliminary diagnosis, keyed out at the fifth order logic domain. No further test is necessary.

Continuing along this logic chain in the associated fifth order logic domain, where the fifth order logic test solves that the extra P-waves do not appear to be saw-toothed and/or flutter waves, then a sixth order logic domain opens for a sixth order logic test in the alternative to determine whether or not the PR interval is constant or variable. Where the sixth order logic test solves that the PR interval is constant, then 2^{nd} degree type II is the preliminary diagnosis, keyed out at the sixth order logic domain. No further test is necessary.

Alternatively, where the sixth order logic test solves that the PR interval is variable, a seventh order logic domain opens to test whether the QRS complex has a width of ≤0.11 seconds (<3 mm) or, alternatively, a width of ≥0.12 seconds (≥3 mm). If the seventh order logic test solves that the QRS complex has a width of ≤0.11 seconds, then proximal 3^{rd} degree is the preliminary diagnosis keyed out at the seventh order logic domain. No further test is necessary. Alternatively, if the seventh order logic test solves that the QRS complex has a width of ≥0.12 seconds, then distal 3^{rd} degree is the preliminary diagnosis. No further test is necessary.

Returning now to the fourth order logic test of whether there is >1 P-wave per QRS, solving in the alternative (at most 1 P-wave per QRS) opens a fifth order logic domain to test whether or not there are three or more different shaped P-waves on the tracing. If the fifth order logic test solves that there are at least 3 different shaped P-waves on the tracing, then the preliminary diagnosis is wandering atrial pacemaker, keyed out at the fifth order logic domain. No further test is necessary.

Alternatively, if the fifth order logic test solves that there are not three or more different shaped P-waves evident on the tracing, a sixth order logic domain opens to test location of the P-waves in the alternative. Where the sixth order logic test solves that the P-waves are located after the QRS complex, accelerated junctional rhythm is the preliminary diagnosis, keyed out at the sixth order logic domain. No further test is necessary. Alternatively, where the sixth order logic test solves that the P-waves are located in front of the QRS complex, then a seventh order logic domain opens wherein the PR interval controls between three possible mutually exclusive outcomes: viz., a PR interval of ≤0.11 seconds results in a preliminary diagnosis of accelerated junctional rhythm. No further test is necessary. A PR interval of 0.12-020 seconds results in a preliminary diagnosis of sinus rhythm. No further test is necessary. And a PR interval of ≥0.21 seconds results in a preliminary diagnosis of sinus rhythm with 1^{st} degree.

Thus, all potential diagnoses for regular heart rates of 60-99 bpm are mutually excluded by following the steps of the present method to present a single preliminary diagnosis by test selecting in the alternative.

The orders of logic domains and associated logic tests under regular rhythm domain solving to 60-99 bpm are summarized below in Table 4.

TABLE 4
Orders of Logic Domains Under Rate 60-99 bpm

| ORDER | TEST | |
|---|---|---|
| III | a. | Presence of P-waves: IV(a) |
| | b. | Absence of P-waves: IV (b) |
| IV(a) | a. | Width of QRS complex ≥012 sec: ACCELARATED |
| | | IDIOVENTRICULAR RHYTHM |
| | b. | Width of QRS complex ≤0.11 sec: ACCELARATED |
| | | JUNCTIONAL RHYTHM |
| IV(b) | a. | >1 P-wave/QRS: V(a) |
| | b. | 1 P-wave/QRS: V(b) |
| V(a) | a. | Extra P-waves are saw-toothed and/ |
| | | or flutter waves: ATRIAL FLUTTER |
| | b. | Extra P-waves are not saw-toothed |
| | | and/or flutter: VI(b)(i) |
| V(b) | a. | There are at least 3 different shaped |
| | | P-waves: WANDERING ATRIAL PACEMAKER |
| | b. | There are less than 3 different shaped P-waves: |
| | | VI(b)(ii) |
| VI(b)(i) | a. | PR interval is constant: 2^{ND} DEGREE TYPE II |
| | b. | PR interval is variable: VII(b)(i) |
| VI(b)(ii) | a. | P-waves located after QRS: ACCELARATED |
| | | JUNCTIONAL RHYTHM |
| | b. | P-waves located before QRS: VII(b)(ii) |
| VII(b)(i) | a. | Width of QRS complex ≥012 sec: DISTAL |
| | | 3^{RD} DEGREE |
| | b. | Width of QRS complex ≤0.11 sec: PROXIMAL |
| | | 3^{RD} DEGREE |
| VII(b)(ii) | a. | PR interval is ≤0.11 sec: ACCELARATED |
| | | JUNCTIONAL RHYTHM |
| | b. | PR interval is 0.12-0.20 sec: SINUS RHYTHM |
| | c. | PR interval is ≥0.21 sec: SINUS RHYTHM |
| | | WITH 1^{ST} DEGREE |

FIG. 7: III Order Logic Domain: Rate 100-149 bpm

In like manner, where the cardiac cycle is regular and rate is between 100 and 149 bpm, a third order logic domain consists of presence or absence of P-waves. Where the third order logic test solves to no P-waves apparent, then the width of the QRS complex presents a fourth order logic domain to key between alternative preliminary diagnoses: ventricular tachycardia (QRS complex width is ≥0.12 seconds), or junctional tachycardia (QRS complex ≤0.11 seconds). No further test is necessary.

Alternatively, where the third order logic test solves that there are P-waves operative within the third order logic domain, then a fourth order logic domain opens to test whether or not there are more than one P-wave per QRS complex. Where the fourth order logic test solves that there are more than one P-wave per QRS complex, a fifth order logic domain opens to test whether or not the extra P-waves are saw-toothed and/or flutter waves (that is, evidence of looped electrical activity to depolarize the atrium). Where the fifth order logic tests solves that the extra P-waves are saw-toothed and/or flutter waves, then atrial flutter is the preliminary diagnosis keyed out at the fifth order logic domain. No further test is necessary.

Continuing along this logic chain in the associated fifth order logic domain in the alternative, where the fifth order logic test solves that the extra P-waves do not appear to be saw-toothed and/or flutter waves, then a sixth order logic domain opens for a sixth order logic test in the alternative to determine whether or not the PR interval is constant or variable. If the sixth order logic test solves that the PR interval is constant, then 2^{nd} degree type II is the preliminary diagnosis keyed out at the sixth order logic domain and no further test is necessary.

If, however, the sixth order logic test solves that the PR interval is variable, then a seventh order logic domain opens to test whether or not the QRS complex has a width of ≤0.11 seconds (<3 mm) or, alternatively, a width of ≥0.12 seconds (≥3 mm). Where the seventh order logic test solves that the QRS complex has a width of ≤0.11 seconds, then proximal 3^{rd} degree is the preliminary diagnosis keyed out at the seventh order logic domain and no further test is necessary. Alternatively, if the seventh order logic test solves that the QRS complex has a width of ≥0.12 seconds, then distal 3^{rd} degree is the preliminary diagnosis at the seventh order logic domain. No further test is necessary.

Returning now to the fourth order logic test of whether or not there is >1 P-wave per QRS, and now solving in the alternative (at most 1 P-wave per QRS), a fifth order logic domain arises to test whether or not there are three or more different shaped P-waves on the tracing. If the fifth order logic test solves that there are at least 3 different shaped P-waves on the tracing, then the preliminary diagnosis is wandering atrial pacemaker keyed out at the fifth order logic domain: No further test is necessary.

Alternatively, where the fifth order logic test solves that there are not three or more different shaped P-waves evident on the tracing, a sixth order logic domain opens to test between locations of the P-waves in the alternative. Where the sixth order logic test solves that the P-waves are located after the QRS complex, junctional tachycardia is the preliminary diagnosis keyed out at the sixth order logic domain and no further test is necessary. Alternatively, where the sixth order logic test solve that the P-waves are located in front of the QRS complex, then a seventh order logic domain opens wherein the PR interval controls between three possible mutually exclusive outcomes: viz., a PR interval of ≤0.11 seconds results in a preliminary diagnosis of junctional tachycardia keyed out at the seventh order logic domain. No further test is necessary. A PR interval of 0.12-020 seconds results in a preliminary diagnosis of sinus tachycardia. No further test is necessary. And a PR interval of ≥0.21 seconds results in a preliminary diagnosis of sinus tachycardia with 1^{st} degree.

Thus, all potential diagnoses for regular heart rates of 100-149 bpm are mutually excluded by following the steps of the present method to present a single preliminary diagnosis by logic tests selecting in the alternative.

The orders of logic domains and associated logic tests under the regular rhythm domain solving to 100-149 bpm are summarized in Table 5.

TABLE 5
Orders of Logic Domains Under Rate 100-149 bpm

| ORDER | TEST | |
|---|---|---|
| III | a. | Presence of P-waves: IV(a) |
| | b. | Absence of P-waves: IV (b) |
| IV(a) | a. | Width of QRS complex ≥012 sec: VENTRICULAR |
| | | TACHYCARDIA |
| | b. | Width of QRS complex ≤0.11 sec: JUNCTIONAL |
| | | TACHYCARDIA |
| IV(b) | a. | >1 P-wave/QRS: V(a) |
| | b. | 1 P-wave/QRS: V(b) |
| V(a) | a. | Extra P-waves are saw-toothed and/or flutter |
| | | waves: ATRIAL FLUTTER |
| | b. | Extra P-waves are not saw-toothed and/or |
| | | flutter: VI(b)(i) |
| V(b) | a. | There are at least 3 different shaped |
| | | P-waves: WANDERING ATRIAL PACEMAKER |
| | b. | There are less than 3 different shaped |
| | | P-waves: VI(b)(ii) |
| VI(b)(i) | a. | PR interval is constant: 2^{ND} DEGREE TYPE II |
| | b. | PR interval is variable: VII(b)(i) |
| VI(b)(ii) | a. | P-waves located after QRS: JUNCTIONAL |
| | | TACHYCARDIA |
| | b. | P-waves located before QRS: VII(b)(ii) |
| VII(b)(i) | a. | Width of QRS complex ≥012 sec: DISTAL |
| | | 3^{RD} DEGREE |
| | b. | Width of QRS complex ≤0.11 sec: PROXIMAL |
| | | 3^{RD} DEGREE |
| VII(b)(ii) | a. | PR interval is ≤0.11 sec: JUNCTIONAL |
| | | TACHYCARDIA |
| | b. | PR interval is 0.12-0.20 sec: SINUS |
| | | TACHYCARDIA |
| | c. | PR interval is ≥0.21 sec: SINUS TACHYCARDIA |
| | | WITH 1^{ST} DEGREE |

FIG. 8: III Order Logic Domain: Rate >150 bpm

Where the cardiac cycle is regular and the rate is >150 bpm a third order logic domain presents alternative diagnoses controlled by the width of the QRS complex. Where the width of the QRS complex is ≤0.11 seconds, supraventricular tachycardia is the preliminary diagnosis. No further test is necessary. Alternatively, when the width of the QRS complex is ≥0.12 seconds, ventricular tachycardia is the preliminary diagnosis. No further test is necessary.

Thus, all potential diagnoses for regular heart rates of >150 bpm are mutually excluded by following the steps of the present method and a single preliminary diagnosis is presented by selecting in the alternative. The orders of logic domains and associated logic tests are summarized in Table 6.

TABLE 6
Orders of Logic Domains Under Rate >150 bpm

| ORDER | TEST | |
|---|---|---|
| III | a. | Width of QRS complex ≥012 sec: VENTRICULAR |
| | | TACHYCARDIA |
| | b. | Width of QRS complex ≤0.11 sec: JUNCTIONAL |
| | | TACHYCARDIA |

FIG. 9: II Order Logic Domain: Irregular Rhythm

Where the first order logic test determines the rhythm is irregular, a second order logic domain opens to test in the alternative whether or not the irregularity is possibly the result of premature contractions of the atria (PACs), junction (PJCs), or the ventricles (PVCs). If the irregularity is possibly the result of PACs, PJCs, or PVCs, then a third order logic domain opens to solve in the alternative whether or not there are P-waves associated with the possible PACs, PJCs, or PVCs. Alternatively, if the irregularity is not possibly the result of PACs, PJCs, or PVCs, then a third order logic domain opens to solve in the alternative whether or not QRS complexes are identifiable.

If the third order logic test solves that P-waves are associated with the possible PACs, PJCs, and PVCs, then a fourth order logic domain opens to determine alternative locations of the P-waves, either in front of the QRS or after the QRS. If the fourth order logic test solves that the P-waves are located after the QRS, then PJCs is the preliminary diagnosis, keyed out at the fourth order logic domain, and no further test is necessary. If the fourth order logic test solves that the P-waves are located in front of the QRS, then a fifth order logic domain opens to solve in the alternative whether the PR interval is ≤0.11 seconds or between 0.12 and 0.20 seconds. If the fifth order logic test solves that the PR interval is ≤0.11 seconds, then PJCs is the preliminary diagnosis, keyed out at the fifth order logic domain, and no further test is necessary. Alternatively, if the fifth order logic test solves that the PR interval is 0.12-0.20 seconds, then PACs is the preliminary diagnosis, keyed out at the fifth order logic domain, and no further test is necessary.

Returning to the third order logic test to solve in the third order logic domain that possible PACs, PJCs, and PVCs do not have associated P-waves, then a fourth order logic domain opens to solve in the alternative whether the width of the QRS in the possible PACs, PJCs, and PVCs, is ≤0.11 seconds or ≥0.12 seconds. If the fourth order logic test solves that the width of the associated QRS is ≤0.11 seconds, then PJCs is the preliminary diagnosis and no further test is necessary. Alternatively, if the fourth order logic test solves that the width of the associated QRS is ≥0.12 seconds, then PVCs is the preliminary diagnosis and no further test is necessary.

If there QRS complexes are not identifiable, then ventricular fibrillation is the preliminary diagnosis, keyed out at the third order logic domain and no further test is necessary. Alternatively, if there are QRS complexes identifiable, then the test opens a fourth order logic domain to solve in the alternative whether or not the irregularity is caused by the presence of pause(s).

If the fourth order logic test solves that the irregularity is possibly caused by pause(s), then a fifth order logic domain opens to solve in the alternative whether the PR interval is between 0.12 and 0.20 seconds or ≥0.21 seconds. If the fifth order logic test solves to PR interval is between 0.12 and 0.20 seconds, then a sixth order logic domain opens to solve in the alternative whether or not, marching across the pause, the RR interval matches when the rhythm resumes after the pause. If the sixth order logic test solves that the RR interval does match, the sinus exit block is the preliminary diagnosis keyed out at the sixth order logic domain and no further test is necessary. Alternatively, if the sixth order logic test determines the RR interval does not match, then sinus arrest is the preliminary diagnosis keyed out at the sixth order logic domain and no further test is necessary.

Where the fifth order logic test solves to PR interval ≥0.21 seconds, then a sixth order logic domain opens to solve in the alternative whether or not, marching across the pause, the RR interval matches when the rhythm resumes after the pause. If the sixth order logic test solves that the RR interval does match, the sinus exit block with 1^{st} degree is the preliminary diagnosis keyed out at the sixth order logic domain and no further test is necessary. Alternatively, if the sixth order logic test determines the RR interval does not match, then sinus arrest with first degree is the preliminary diagnosis keyed out at the sixth order logic domain and no further test is necessary.

Returning now to the fourth order logic domain of irregularity possibly caused by pauses, where the fourth order logic test solves that the irregularity is not possibly caused by pause(s), then a fifth order logic domain opens to solve in the alternative whether or not P-waves are discernible. If the fifth order logic test solves that there are not any P-waves discernible, then atrial fibrillation is the preliminary diagnosis keyed out at the fifth order logic domain and no further test is necessary. Alternatively, if the fifth order logic test solves that there are P-waves determinable, then a sixth order logic domain opens to solve in the alternative whether or not there are at least three different shaped P-waves.

If the sixth order logic test determines that there are at least three different shaped P-waves, then wandering atrial pacemaker is the preliminary diagnosis keyed out at the sixth order logic domain and no further test is necessary. Alternatively, if the sixth order logic test solves that there are not at least three different shaped P-waves, then a seventh order logic domain opens to solve in the alternative whether or not there is more than one P-wave per QRS complex.

If the seventh order logic test solves to there is not more than one P-wave per QRS complex, then an eight order logic domain opens to solve in the alternative whether the PR interval is either between 0.12 seconds (3 mm) and 0.20 seconds (5 mm) or ≥0.21 seconds (≥5.25 mm). If the eighth order logic test solves to PR interval of 0.12-0.20 seconds, then sinus arrhythmia with 1^{st} degree is the preliminary diagnosis keyed out at the eighth order logic domain and no further test is necessary. Alternatively, if the eighth order logic test solves to PR interval of ≥0.21 seconds, then sinus arrhythmia is the preliminary diagnosis keyed out at the eighth order logic domain and no further test is necessary.

Returning to the seventh order logic domain wherein the seventh order logic test solves that there are more than one P-wave per QRS complex, an eighth order logic domain opens to solve in the alternative whether or not the extra P-waves are saw-toothed and/or flutter waves. If the eight order logic test solves to the extra P-waves are saw-toothed and/or flutter waves, then atrial flutter is the preliminary diagnosis keyed out at the eighth order logic domain and no further test is necessary. Alternatively, if the eight order logic test solves to the extra P-waves are not saw-toothed and/or flutter waves, a ninth order logic domain opens to solve in the alternative whether the PR interval is variable or constant. Where the ninth order logic test solves to the PR interval is variable, 2^{nd} degree type I is the preliminary diagnosis keyed out at the ninth order logic domain and no further test is necessary. Where the ninth order logic test solves to the PR interval is constant, 2^{nd} degree type II is the preliminary diagnosis keyed out at the ninth order logic domain and no further test is necessary.

The orders of logic domains and associated logic tests associated under the first order logic domain solving to irregular rhythm are summarized in Table 7.

TABLE 7
Orders of Logic Domains Under Irregular Rhythm

| ORDER | TEST | |
|---|---|---|
| II | a. | Irregularity possibly caused by PACs, PJCs, |
| | | or PVCs: III(a) |
| | b. | Irregularity not possibly caused by PACs, PJCs, |
| | | or PVCs: III(b) |
| III(a) | a. | P-waves associated with possible PACs, PJCs, |
| | | or PVCs: IV(a) |
| | b. | P-waves not associated with possible PACs, PJCs, |
| | | or PVCs: IV(b) |
| III(b) | a. | QRS complexes are identifiable: IV(c) |
| | b. | QRS complexes are not identifiable: VENTRICULAR |
| | | FIBRILLATION |
| IV(a) | a. | P-waves before QRS for possible PACs, PJCs, |
| | | or PVCs: V(a) |
| | b. | P-waves after QRS for possible PACs, PJCs, |
| | | or PVCs: PJCs |
| IV(b) | a. | Width of QRS for possible PACs, PJCs, |
| | | or PVCs ≤0.11 sec: PJCs |
| | b. | Width of QRS for possible PACs, PJCs, |
| | | or PVCs ≥0.12 sec: PVCs |
| IV(c) | a. | Irregularity possibly caused by pause(s): V(b) |
| | b. | Irregularity not possibly caused by pause(s):V(c) |
| V(a) | a. | PR interval ≤0.11 sec: PJCs |
| | b. | PR interval ≥0.12 sec: PACs |
| V(b) | a. | PR interval is 0.12-0.20 sec: VI(a) |
| | b. | PR interval is ≥0.21 sec: VI(b) |
| V(c) | a. | P-waves are discernible: VI(c) |
| | b. | P-waves are not discernible: ATRIAL FIBRILLATION |
| VI(a) | a. | RR interval matches marching across the pause: SINUS |
| | | EXIT BLOCK |
| | b. | RR interval does not match marching across the |
| | | pause: SINUS ARREST |
| VI(b) | a. | RR interval matches marching across the pause: SINUS |
| | | EXIT BLOCK WITH 1^{ST} DEGREE |
| | b. | RR interval does not match marching across the |
| | | pause: SINUS ARREST WITH 1^{ST} DEGREE |
| VI(c) | a. | ≥3 different shapes of P-waves: WANDERING |
| | | ATRIAL PACEMAKER |
| | b. | <3 different shapes of P-waves: VII(a) |
| VII(a) | a. | >1 P-wave per QRS: VIII(a) |
| | b. | ≤1 P-wave per QRS: VIII(b) |
| VIII(a) | a. | Extra P-waves appear saw-toothed and/ |
| | | or flutter waves: ATRIAL FLUTTER |
| | b. | Extra P-waves do not appear saw-toothed and/ |
| | | or flutter waves: IX |
| VIII(b) | a. | QRS interval 0.12-0.20 sec: SINUS ARRHYTHMIA |
| | b. | QRS interval ≥0.21 sec: SINUS ARRHYTHMIA |
| | | WITH 1^{ST} DEGREE |
| IX | a. | PR interval is variable: 2^{ND} DEGREE TYPE I |
| | b. | PR interval is constant: 2^{ND} DEGREE TYPE II |

What is claimed is:

1. A method for rapidly diagnosing a heart condition of a patient from associated electrocardiogram (“ECG”) data, the method comprising the steps of:
in a first order logic domain, conducting a first order logic test to solve in the alternative between regularity or irregularity of a cardiac rhythm wherein corresponding second order logic domains are opened in the alternative;
in the second order logic domain corresponding to irregularity of the cardiac rhythm, initiating a second order logic test in the alternative to select between successive third, fourth, fifth, sixth, seventh, eighth, and ninth order logic domains wherein associated third, fourth, fifth, sixth, seventh, eighth and ninth order logic tests present a preliminary diagnosis mutually excluded from the group consisting of:
ventricular fibrillation, atrial fibrillation, wandering atrial pacemaker, sinus arrhythmia, sinus arrhythmia with 1^{st} degree, 2^{nd} degree type I, 2^{nd} degree type II, atrial flutter, sinus arrest, sinus arrest with 1^{st} degree, sinus exit block, sinus exit block with 1^{st} degree, premature atrial contractions (“PACs”), premature ventricular contractions (“PVCs”), and premature junctional contractions (“PJCs”); and
in the second order logic domain corresponding to regular rhythm, initiating a second order logic test to select in the alternative between ranges of heart rates, wherein each of said ranges of heart rates opens a corresponding third order logic domain wherein a third order logic test opens successive fourth, fifth, sixth, and seventh order logic domains, and wherein corresponding fourth, fifth, sixth, and seventh order logic tests present a preliminary diagnosis mutually excluded from the group consisting of:
idioventricular rhythm, accelerated idioventricular rhythm, ventricular tachycardia, supraventricular tachycardia, junctional rhythm, accelerated junctional rhythm, junctional tachycardia, sinus rhythm, sinus rhythm with 1^{st} degree, sinus bradycardia, sinus bradycardia with 1^{st} degree, sinus tachycardia, sinus tachycardia with 1^{st} degree, wandering atrial pacemaker, atrial flutter, 2^{nd} degree type II, proximal 3^{rd} degree, distal 3^{rd} degree.

2. The method of claim 1 wherein the second order logic domain under regularity of the cardiac rhythm comprises the steps of:
running a second order logic test to determine whether the beats per minute (bpm) are:
≤40 bpm;
from 41 to 59 bpm;
from 60 to 99 bpm;
from 100 to 149 bpm; or
≥150 bpm;
wherein determination of the bpm opens the corresponding third order logic domain wherein the corresponding third order logic test is initiated.

3. The method of claim 1 wherein the second order logic test operative in the second order logic domain under irregularity of the cardiac rhythm comprises the steps of:
solving presence or absence of any or premature atrial contractions (“PACs”), premature junctional contractions (“PJCs”), or premature ventricular contractions (“PVCs”);
wherein presence or absence of PACs, PJCs, or PVCs, opens the third order logic domain wherein the third order logic test is initiated.

4. The method of claim 2 wherein the third order logic test operative in the third order logic domain under heart rate ≥150 bpm comprises the steps of:
solving in the alternative whether the width of the QRS complex is either ≥0.12 seconds or ≤0.11 seconds;
determining a preliminary diagnosis of ventricular tachycardia where the QRS complex is ≥0.12 seconds; and
determining a preliminary diagnosis of supraventricular tachycardia where the QRS complex is ≥0.11 seconds.

5. The method of claim 2 wherein the third order logic test operative in the third order logic domain under rate ≤40 bpm comprises the step of:
solving in the alternative for presence of absence of P-waves;
wherein presence or absence of P-waves opens the fourth order logic domain wherein the fourth order logic test is initiated.

6. The method of claim 5 wherein the fourth order logic test operative in the fourth order logic domain under absence of P-waves comprises the steps of:
solving in the alternative whether the width of the QRS complex is ≥0.12 seconds or ≤0.11 seconds;
determining a preliminary diagnosis of idioventricular rhythm where the QRS complex is ≥0.12 seconds; and
determining a preliminary diagnosis of junctional rhythm where the QRS complex is ≤0.11 seconds.

7. The method of claim 5 wherein the fourth order logic test operative in the fourth order logic domain under presence of P-waves comprises the step of:
solving whether or not there are more than one P-wave per QRS complex;
wherein presence of absence of more than one P-waves per QRS complex opens the fifth order logic domain wherein the fifth order logic test is initiated.

8. The method of claim 7 wherein the fifth order logic test operative in the fifth order logic domain under presence of more than one P-waves per QRS complex comprises the steps of:
solving whether or not the extra P-waves are saw-toothed and/or flutter waves;
presenting a preliminary diagnosis of atrial flutter where the P-waves are saw-toothed and/or flutter waves; and
opening a sixth order logic domain where the extra P-waves do not appear to be saw-toothed and/or flutter waves.

9. The method of claim 8 wherein the sixth order logic test operative in the sixth order logic domain comprises the steps of:
determining in the PR interval is constant or variable;
diagnosing 2^{nd} degree type II were the PR interval is constant; and
initiating the seventh order logic test operative within the corresponding seventh order logic domain where the PR interval is variable.

10. The method of claim 9 wherein the seventh order logic test operative within the seventh order logic domain comprises the steps of:
determining whether the width of the QRS complex is either ≥0.12 seconds or ≤0.11 seconds;
diagnosing distal 3^{rd} degree where the width of the QRS complex is ≥0.12 seconds; and
diagnosing proximal third degree where the width of the QRS complex is ≤0.11 seconds.

11. The method of claim 7 wherein the fifth order logic test operative in the fifth order logic domain under absence of more than one P-wave per QRS complex comprises the steps of:
solving in the alternative between:
presence of at least three different shaped P-waves; or
absence of at least 3 different shaped P-waves; and
diagnosing wandering atrial pacemaker where there is presence of at least 3 different shaped P-waves; or
opening the sixth order logic domain where there is absence of at least 3 different shaped P-waves.

12. The method of claim 11 wherein the sixth order logic test operative in the sixth order logic domain comprises the steps of:
solving in the alternative whether:
the P-waves are in front of the QRS complex; or
after the QRS complex; and
presenting a preliminary diagnosis of junctional rhythm where the P-waves occur after the QRS complex; or
opening a seventh order logic domain where the P-waves occur in front of the QRS complex.

13. The method of claim 12 wherein the seventh order logic test operative within the seventh order logic domain comprises the steps of:
determining whether the PR interval is ≤0.11 seconds, between 0.12 seconds and 0.20 seconds, or ≥0.21 seconds;
diagnosing junctional rhythm where the PR interval is ≤0.11 seconds;
diagnosing sinus bradycardia where the PR interval is between 0.12 seconds and 0.20 seconds; and
diagnosing sinus bradycardia with 1^{st} degree where the PR interval is ≥0.21 seconds.

14. The method of claim 2 wherein the third order logic test operative in the third order logic domain under rate of 41-59 bpm comprises the step of:
determining presence of absence of P-waves;
wherein the presence or absence of P-waves initiates a fourth order logic test operative within a corresponding fourth order logic domain.

15. The method of claim 14 wherein the fourth order logic test operative in the fourth order logic domain under absence of P-waves comprises the steps of:
determining whether the width of the QRS complex is either ≥0.12 seconds or ≤0.11 seconds;
presenting a preliminary diagnosis of accelerated idioventricular rhythm where the QRS complex is ≥0.12 seconds; and
presenting a preliminary diagnosis of junctional rhythm where the QRS complex is ≤0.11 seconds.

16. The method of claim 14 wherein the fourth order logic test operative in the fourth order logic domain under presence of P-waves comprises the step of:
determining whether there are more than one P-wave per QRS complex;
wherein presence of absence of more than one P-wave per QRS complex opens the fifth order logic domain wherein the fifth order logic test is initiated.

17. The method of claim 16 wherein the fifth order logic test operative in the fifth order logic domain under presence of more than one P-wave per QRS complex comprises the steps of:
determining if the extra P-waves appear to be saw-toothed and/or flutter waves;
diagnosing atrial flutter where the extra P-waves appear to be saw-toothed and/or flutter waves; and
opening the sixth order logic domain where the P-waves do not appear to be saw-toothed and/or flutter waves.

18. The method of claim 17 wherein the sixth order logic test operative in the sixth order logic domain comprises the steps of:
determining whether the PR interval is constant or variable;
diagnosing second degree type II were the PR interval is constant; and
opening the seventh order logic test where the PR interval is variable.

19. The method of claim 18 wherein the seventh order logic test operative within the seventh order logic domain comprises the steps of:
determining whether the width of the QRS complex is either ≥0.12 seconds or ≤0.11 seconds;
presenting a preliminary diagnosis of distal third degree where the width of the QRS complex is ≥0.12 seconds; and
presenting a preliminary diagnosis of proximal third degree where the width of the QRS complex is ≤0.11 seconds.

20. The method of claim 16 wherein the fifth order logic test operative in the fifth order logic domain under absence of more than one P-wave per QRS complex comprises the steps of:
determining presence of absence of at least three different shaped P-waves;
diagnosing wandering atrial pacemaker where there is presence of at least three different shaped P-waves; and
opening the sixth order logic domain where there is absence of at least 3 different shaped P-waves.

21. The method of claim 20 wherein the fifth order logic test operative in the sixth order logic domain comprises the steps of:
determining whether the P-waves occur in front of the QRS complex or after the QRS complex;
presenting a preliminary diagnosis of junctional rhythm where the P-waves occur after the QRS complex; and
opening the seventh order logic domain where the P-waves occur in front of the QRS complex.

22. The method of claim 21 wherein the seventh order logic test operative within the seventh order logic domain comprises the steps of:
determining whether the PR interval is one of:
≤0.11 seconds;
between 0.12 seconds and 0.20 seconds; or
≥0.21 seconds; and
presenting a preliminary diagnosis of junctional rhythm where the PR interval is ≤0.11 seconds; or
presenting a preliminary diagnosis of sinus bradycardia where the PR interval is between 0.12 seconds and 0.20 seconds; or
presenting a preliminary diagnosis of sinus bradycardia with 1^{st} degree where the PR interval is ≥0.21 seconds.

23. The method of claim 2 wherein the third order logic test operative in the third order logic domain under rate of 60-99 bpm comprises the step of:
determining presence of absence of P-waves;
wherein the presence or absence of P-waves opens the fourth order logic domain.

24. The method of claim 23 wherein the fourth order logic test operative in the fourth order logic domain under absence of P-waves comprises the steps of:
determining whether the width of the QRS complex is either ≥0.12 seconds or ≤0.11 seconds;
presenting a preliminary diagnosis of accelerated idioventricular rhythm where the QRS complex is ≥0.12 seconds; and
presenting a preliminary diagnosis of accelerated junctional rhythm where the QRS complex is ≤0.11 seconds.

25. The method of claim 23 wherein the fourth order logic test operative in the fourth order logic domain under presence of P-waves comprises the step of:
determining whether there is more than one P-wave per QRS complex;
wherein presence of absence of more than one P-wave per QRS complex opens the fifth order logic domain.

26. The method of claim 25 wherein the fifth order logic test operative in the fifth order logic domain under presence of more than one P-wave per QRS complex comprises the steps of:
determining if the extra P-waves appear to be saw-toothed and/or flutter waves;
presenting a preliminary diagnosis of atrial flutter where the extra P-waves appear to be saw-toothed and/or flutter waves; and
opening the sixth order logic domain where the extra P-waves do not appear to be saw-toothed and/or flutter waves.

27. The method of claim 26 wherein the sixth order logic test operative in the sixth order logic domain comprises the steps of:
determining if the PR interval is constant or variable;
presenting a preliminary diagnosis of second degree type II were the PR interval is constant; and
opening the seventh order logic domain where the PR interval is variable.

28. The method of claim 27 wherein the seventh order logic test operative within the seventh order logic domain comprises the steps of:
determining whether the width of the QRS complex is either ≥0.12 seconds or ≤0.11 seconds;
presenting a preliminary diagnosis of distal third degree where the width of the QRS complex is ≥0.12 seconds; and
presenting a preliminary diagnosis of proximal third degree where the width of the QRS complex is ≤0.11 seconds.

29. The method of claim 25 wherein the fifth order logic test operative in the fifth order logic domain under absence of more than one P-wave per QRS complex comprises the steps of:
determining presence of absence of at least three different shaped P-waves;
diagnosing wandering atrial pacemaker where there is presence of at least three different shaped P-waves; and
opening the sixth order logic domain where there is absence of at least three different shaped P-waves.

30. The method of claim 39 wherein the sixth order logic test operative in the sixth order logic domain comprises the steps of:
determining whether the P-waves are in front of the QRS complex or after the QRS complex;
presenting a preliminary diagnosis of accelerated junctional rhythm where the P-waves are after the QRS complex; and
opening the seventh order logic domain where the P-waves are before the QRS complex.

31. The method of claim 30 wherein the seventh order logic test operative within the seventh order logic domain comprises the steps of:
determining whether the PR interval is one of:
≤0.11 seconds;
between 0.12 seconds and 0.20 seconds; or
≥0.21 seconds;
presenting a preliminary diagnosis of accelerated junctional rhythm where the PR interval is ≤0.11 seconds;
presenting a preliminary diagnosis of sinus rhythm where the PR interval is between 0.12 seconds and 0.20 seconds; and
presenting a preliminary diagnosis of sinus rhythm with 1^{st} degree where the PR interval is ≥0.21 seconds.

32. The method of claim 2 wherein the third order logic test operative in the third order logic domain under rate of 100-149 bpm comprises the step of:
determining presence of absence of P-waves;
wherein the presence or absence of P-waves opens the fourth order logic domain.

33. The method of claim 32 wherein the fourth order logic test operative in the fourth order logic domain under absence of P-waves comprises the step of:
determining whether the width of the QRS complex is ≥0.12 seconds or ≤0.11 seconds;
presenting a preliminary diagnosis of ventricular tachycardia where the QRS complex is ≥0.12 seconds; and
presenting a preliminary diagnosis of junctional tachycardia where the QRS complex is ≤0.11 seconds.

34. The method of claim 32 wherein the fourth order logic test operative in the fourth order logic domain under presence of P-waves comprises the step of:
determining whether there are more than one P-wave per QRS complex;
wherein presence of absence of more than one P-waves per QRS complex opens the fifth order logic domain.

35. The method of claim 34 wherein the fifth order logic test operative in the fifth order logic domain under presence of at least 2 P-waves per QRS complex comprises the step of:
determining if the P-waves appear to be saw-toothed and/or flutter waves;
presenting a preliminary diagnosis of atrial flutter where the P-waves appear to be saw-toothed and/or flutter waves; and
opening the sixth order logic domain where the P-waves do not appear to be saw-toothed and/or flutter waves.

36. The method of claim 35 wherein the sixth order logic test operative in the sixth order logic domain comprises the steps of:
determining in the PR interval is constant or variable;
presenting a preliminary diagnosis of second degree type II were the PR interval is constant; and
opening the seventh order logic domain where the PR interval is variable.

37. The method of claim 36 wherein the seventh order logic test operative within the seventh order logic domain comprises the steps of:
determining whether the width of the QRS complex is ≥0.12 seconds or ≤0.11 seconds;
presenting a preliminary diagnosis of distal third degree where the width of the QRS complex is ≥0.12 seconds; and
presenting a preliminary diagnosis of proximal third degree where the width of the QRS complex is ≤0.11 seconds.

38. The method of claim 34 wherein the fifth order logic test operative in the fifth order logic domain under absence of at least 2 P-waves per QRS complex comprises the steps of:
determining presence of absence of at least three different shaped P-waves;
presenting a preliminary diagnosis of wandering atrial pacemaker where there is presence of at least three different shaped P-waves; and
opening the sixth order logic domain where there is absence of at least 3 different shaped P-waves.

39. The method of claim 38 wherein the sixth order logic test operative in the sixth order logic domain comprises the steps of:
determining whether the P-waves are either in front of the QRS complex or after the QRS complex;
presenting a preliminary diagnosis of junctional tachycardia where the P-waves are after the QRS complex; and
opening the seventh order logic domain where the P-waves are before the QRS complex.

40. The method of claim 39 wherein the seventh order logic test operative within the seventh order logic domain comprises the steps of:
determining whether the PR interval is one of:
≤0.11 seconds;
between 0.12 seconds and 0.20 seconds; or
≥0.21 seconds; and
presenting a preliminary diagnosis of junctional tachycardia where the PR interval is ≤0.11 seconds;
presenting a preliminary diagnosis of sinus tachycardia where the PR interval is between 0.12 seconds and 0.20 seconds; and
presenting a preliminary diagnosis of sinus tachycardia with 1^{st} degree where the PR interval is ≥0.21 seconds.

41. The method of claim 3 wherein the third order logic test operative in the third order logic domain under presence of PACs, PJCs, or PVCs, comprises the step of:
determining presence of absence of P-waves associated with the PACS, PJCs, or PVCs;
wherein the presence or absence of P-waves opens the fourth order logic domain.

42. The method of claim 41 wherein the fourth order logic test operative within the fourth order logic domain under absence of P-waves associated with the PACs, PJCs, or PVCs comprises the steps of:
determining whether the width of a QRS complex is either ≤0.11 seconds or ≥0.12 seconds;
presenting a preliminary diagnosis of PJCs where the QRS complex is ≤0.11 seconds; and
presenting a preliminary diagnosis of PVCs where the QRS complex is ≥0.12 seconds.

43. The method of claim 41 wherein the fourth order logic test operative within the fourth order logic domain under presence of P-waves associated with the PACs, PJCs, or PVCs comprises the steps of:
determining the position of the P-waves associated with the PACs, PJCs, or PVCs as either in front of the QRS complex or after the QRS complex;
presenting a preliminary diagnosis of PJCs where the P-waves associated with the PACs, PJCs, or PVCs are determined to be after the QRS complex; and
opening the fifth order logic domain where the position of the said P-waves is in front of the QRS complex.

44. The method of 43 wherein the fifth order logic test operative within the fifth order logic domain comprises the steps of:
determining whether a PR interval is either ≤0.11 seconds or between 0.12 and 0.20 seconds;
presenting a preliminary diagnosis of PJCs where the PR interval is ≤0.11 seconds; and
presenting a preliminary diagnosis of PACs where the PR interval is between 0.12 to 0.20 seconds.

45. The method of claim 3 wherein the third order logic test operative in the third order logic domain under absence of PACs, PJCs, or PVCs, comprises the steps of:
determining presence or absence of QRS complexes;
presenting a preliminary diagnosis of ventricular fibrillation where there is absence of the QRS complexes; and
opening the fourth order logic domain where there is presence of QRS complexes.

46. The method of claim 45 wherein the fourth order logic test operative within the fourth order logic domain comprises the step of:
determining whether the irregularity of the cardiac rhythm is possibly caused by pauses;
wherein the presence or absence of pauses opens the fifth order logic domain.

47. The method of claim 46 wherein the fifth order logic test operative within the fifth order logic domain under the presence of pauses comprises the step of:
determining whether the PR interval is either between 0.12 and 0.20 seconds or >0.20 seconds;
wherein corresponding length of the PR interval determined in the fifth order logic domain opens the sixth order logic domain.

48. The method of 47 wherein the sixth order logic test operative within the sixth order logic domain under the PR interval between 0.12 and 0.20 seconds comprises the steps of:
determining the whether an RR interval across the pause matches the RR interval when the rhythm resumes after the pause;
presenting a preliminary diagnosis of sinus arrest where the said RR intervals do not match; and
presenting a preliminary diagnosis of sinus exit block where the said RR intervals do match.

49. The method of 47 wherein the sixth order logic test operative within the sixth order logic domain under the PR interval >0.20 seconds comprises the steps of:
determining whether an RR interval across the pause matches the RR interval when the rhythm resumes after the pause;
presenting a preliminary diagnosis of sinus arrest with first degree where the said RR intervals do not match; and
presenting a preliminary diagnosis of sinus exit block with first degree where the said RR intervals do match.

50. The method of claim 46 wherein the fifth order logic test operative within the fifth order logic domain under the absence of pauses comprises the steps of:
determining the presence or absence of P-waves; and
presenting a preliminary diagnosis of atrial fibrillation in the absence of P-waves;
opening the sixth order logic domain operative in the presence of P-waves.

51. The method of claim 50 wherein the sixth order logic test operative within the sixth order logic domain comprises the steps of:
determining the presence or absence of at least three different shaped P-waves;
presenting a preliminary diagnosis of wandering atrial pacemaker in the presence of at least three different shaped P-waves; and
opening the seventh order logic domain operative in the absence of at least three different shaped P-waves.

52. The method of claim 51 wherein the seventh order logic test operative within the seventh order logic domain comprises the step of:
determining the presence or absence of more than one P-wave per QRS complex;
wherein the presence or absence of more than one P-wave per QRS complex opens the eighth order logic domain.

53. The method of claim 52 wherein the eighth order logic test operative within the eighth order logic domain under the absence of at least two P-waves per QRS complex comprises the steps of:
determining either the presence of a PR interval ≤0.21 seconds or a PR interval between 0.12 and 0.20 seconds;
presenting a preliminary diagnosis of sinus arrhythmia with first degree in the presence of the PR interval ≤0.21 seconds; and
presenting a preliminary diagnosis of sinus arrhythmia in the presence of the PR interval between 0.12 and 0.20 seconds.

54. The method of claim 52 wherein the eighth order logic test operative within the eighth order logic domain under the presence of at least two P-waves per QRS complex comprises the steps of:
determining the presence or absence of sawtooth and/or flutter P-waves; and
presenting a preliminary diagnosis of atrial flutter in the presence of sawtooth and/or flutter P-waves;
opening the ninth order logic domain in the absence of sawtooth and/or flutter P-waves.

55. The method of claim 52 wherein the ninth order logic test operative within the ninth order domain comprises the steps of:
determining the presence of a variable PR interval;
determining the presence of a constant PF interval;
presenting a preliminary diagnosis of second degree type I in the presence of the variable PR interval; and
presenting a preliminary diagnosis of second degree type II in the presence of the constant PR interval.
